(19) United States
(12) Patent Application Publication
(10) Pub. No.: US 2022/0291527 A1
(43) Pub. Date: Sep. 15, 2022

(54) Eyeglass Frames For Treatment Of Dry Eye Syndrome And Corresponding 3D Printing Systems And Methods

(71) Applicant: The Regents of the University of Colorado, a body corporate, Denver, CO (US)

(72) Inventors: Stephen Petty, Denver, CO (US); Cristos Ifantides, Denver, CO (US); Jahmel Jordon, Denver, CO (US); Jonathan Petty, Lafayette, CO (US); Jeffrey SooHoo, Denver, CO (US)

(73) Assignee: The Regents of the University of Colorado, a body corporate, Denver, CO (US)

(21) Appl. No.: 17/631,959

(22) PCT Filed: Jul. 31, 2020
(86) PCT No.: PCT/US2020/044498

§ 371 Date: Feb. 1, 2022

Related U.S. Application Data
(60) Provisional application No. 62/881,688, filed on Aug. 1, 2019.

(51) Int. Cl. G02C 11/00; A61F 9/02

(52) U.S. Cl. CPC G02C 11/00; A61F 9/026; A61F 2009/021

(57) ABSTRACT

Various embodiments of the present technology generally relate to treatment for dry eyes. More specifically, some embodiments of the present technology relate to eyeglass frames for treatment of dry eye syndrome and corresponding three-dimensional printing systems and methods. In some embodiments an eyewear apparatus includes a frame with a seal configured to engage with the skin of the wearer around a majority of the perimeter of an interior portion of the front of the frame sufficient to keep the humidity around the eye high enough to provide relief from symptoms of dry eye syndrome.

### CROSS-REFERENCE TO RELATED APPLICATIONS

The present application claims priority to U.S. Provisional Application No. 62/881,688 filed Aug. 1, 2019 titled “Eyeglass Frames for Treatment of Dry Eye Syndrome and Corresponding 3D Printing Systems and Methods,” which is incorporated herein by reference in its entirety for all purposes.

### TECHNICAL FIELD

Various embodiments of the present technology generally relate to treatment for dry eyes. More specifically, some embodiments of the present technology relate to eyeglass frames for treatment of dry eye syndrome and corresponding three-dimensional printing systems and methods.

### BACKGROUND

Keratoconjunctivitis sicca (KCS), also known as dry eye syndrome, is a common disease that can have significant impact on a person's visual acuity, daily activities, social and physical functioning, workplace productivity, and other necessary functions. Approximately one out of seven individuals ages sixty-five to eighty-four years reports symptoms of dry eye disease. The prevalence of dry eyes has been estimated to be five to thirty percent in persons over fifty years of age and the percentage is expected to increase. Based on data from the National Health and Wellness Survey, 6.8 percent of the United States adult population (16.4 million individuals) has been diagnosed with dry eye syndrome.

The lacrimal glands, eyelids, and ocular surface (know together as the lacrimal functional unit) are responsible for tear film production and retention. Dry eye syndrome is thought to be caused by a dysfunction in any component involved in tear film production. The tear film of the eye consists of aqueous, mucous, and lipid components that function synergistically to lubricate the ocular surface and reduce evaporation of the tear film layer to the ambient environment. Decreased tear production and/or increased tear evaporation due to lacrimal gland dysfunction or destruction (caused by inflammation) leads to excessive drying of the ocular surface leading to symptoms including dryness, irritation, burning, light sensitivity, and, in severe cases, blurred vision. In extreme cases of dry eye syndrome, permanent damage to visual acuity can occur from corneal scarring. Furthermore, dry eye patients are prone to potentially blinding infections such as bacterial keratitis and experience an increased risk of complications following common procedures such as laser refractive surgery.

Treatment of dry eye syndrome is generally aimed at increasing or supplementing tear production, reducing tear resorption, or reducing ocular surface inflammation through the use of artificial tears, topical cyclosporine, and/or surgical procedures. Few solutions are targeted at protecting the local humidity around the eyes to prevent the evaporative loss of the tear film to the external environment. Of the limited options of present solutions, most are akin to swim goggles or resemble “moisture chambers” that can be fitted to existing glasses in optical shops by trained opticians.

### SUMMARY OF THE INVENTION

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Various embodiments of the present technology generally relate to treatment for dry eyes. More specifically, some embodiments of the present technology relate to eyeglass frames for treatment of dry eye disease and corresponding three-dimensional printing systems and methods. In some embodiments an eyewear apparatus includes a frame with a seal configured to engage the skin of the wearer around a majority, but not all, of the perimeter of an interior portion of the front of the frame sufficient to keep the humidity around the eye at high enough level to provide relief for systems of dry eye syndrome.

In some embodiments, the frame can include a front portion having two eyepieces connected by a bridge. The eyepieces (e.g., openings) are designed to securely hold a right lens and a left lens. A custom seal can be attached (or be directly formed) along a majority of the perimeter of the inside of frame surrounding the eyepieces. For example, the seal may be formed in some embodiments so that only the temporal side is left open allowing for air flow. In some embodiments, the seal can be designed to engage the skin along the face of the user and thereby fill a void between at least a portion of the front of the frame and the face of a user. For example, the seal can be designed to fill a void between a superior aspect of the front of the frame and the face of the user, a void between an inferior aspect of the front of the frame and the face of the user, and a void between a nasal region of the front of the frame and the face of the user. In some examples, a right temporal aspect of the front of the frame and a left temporal aspect of the frame may not be sealed (or at least not entirely sealed) such that the right temporal aspect and the left temporal aspect allow an airflow between the eyewear apparatus and the face.

In accordance with various embodiments, the seal, at least in part, helps maintain an increased humidity level between the eyewear apparatus and the face as compared to the surrounding environment. For example, depending on the external humidity level and the size and shape of the open portions (e.g., on the temporal aspects of the frame), various embodiments may see an internal humidity level range from below ten percent to over ninety-five percent. Moreover, the internal humidity level may vary during the day, based on the location of the individual (e.g., outside vs. inside an office building), perspiration of the individual, materials of the seal and/or frames, placement of the eyewear on the individual's face, and/or other factors. Some embodiments of the eyewear may keep the humidity level between forty percent and a maximum humidity level of ninety percent. In other embodiments, the eyewear may keep the humidity level between twenty percent and sixty percent, thirty percent and eight percent, five percent and twenty percent, five percent and eighty percent, sixty percent and one hundred percent, and/or many other ranges depending on the design, external conditions, individual traits (e.g., perspiration).

In some embodiments, the frame and the seal can be built separately such that the frame includes one or more receiving interfaces (e.g., attachment posts or ridges for press fitting, apertures for screws, etc.). The seal can also include one or more attachment components and be designed to allow for secure fixation and removal from the front of the frame. In this manner, various frames can be created (e.g., using a 3D printer, molds, etc.), and only an interface structure (e.g. a seal) needs to be created, printed, or formed according to the specific facial structure of a wearer. In other embodiments, the frame and the interface surface can both be custom designed for the face of a user.

Embodiments of the present technology can also include computer-readable storage media containing sets of instructions that when executed by one or more processors cause one or more machines to perform the methods, variations of the methods, and other operations described herein.

In some embodiments, a 3D scan of at least a portion of a face can be initiated. Next, a face model of at least a portion of the face can then be generated. The face model may be the same portion of the face from the 3D scan, or a smaller portion of the face from the 3D scan. Then, a model of an interface structure (e.g., seal, ridgeline, skin engaging component can be generated. For example, in some embodiments, the interface structure can be designed to have a profile that fills a void between a superior portion of an eyeglass frame and the face, an inferior portion of the eyeglass frame and the face, and a nasal portion of the eyeglass frame and the model of the face. The face model generated of at least a portion of the face, in some examples, can be a 3D rendering of the face.

In some embodiments, a single 3D scan of the individual wearing a pair of eyeglasses as described in the present disclosure is acquired and used as a singular point of reference for further eyeglass design. In other embodiments, alternatively, a series of 3D scans of the individual's face may be acquired with and without the glasses. First, a high-quality scan of the glasses of choice is acquired. Next, a scan of the individual's face looking forward without the glasses being worn is acquired. Finally, a final scan of the individual's face while wearing the glasses in the desired orientation is acquired. Using the series of scans, a computer system, design software, 3D printer, or the like may be used to generate a pair of eyeglasses.

In various embodiments, facial scans can be acquired in several modalities to reach the same goal of creating 3D printed eyeglasses. For example, multiple scans may be acquired using various 3D-scanning technologies including, but not limited to, contact 3D scanners, time-of-flight or triangulation 3D laser scanners, structured or modulated light 3D scanners, stereoscopic, photometric, silhouette active 3D scanners, existing point clouds, and 3D scans contained in polygon meshes or computer-aided design (CAD) models. In addition, the patient scan data may be reconstructed from medical imaging modalities such as computed tomography (CT) scans and magnetic resonance imaging (MRI). Although CT scans and MRIs do not produce point cloud data similar to other 3D scanning modalities like those mentioned above, 3D volumetric renderings can be constructed using processes such as volume rendering, image segmentation, and image-based meshing, as just a few examples.

Some embodiments provide for a system that includes a 3D facial scanner, a computer system, design software, and/or a 3D printer. The 3D facial scanner can be used to generate a facial scan of at least a portion of a face. The facial scan can be received by the computer system which can execute the design software to generate a model of at least a portion of the face based on the facial scan and a frame that that includes a front that would substantially engage and enclose (e.g., more than 50%, 60%, 70%, 80% or 90%, between 55% and 95%, between 60% and 80%, enough to increase the humidity level, or the like) the area around the eyes of the user during normal wear. For example, the front of the frame may be designed to receive or create a seal having a topographical profile designed to fill (or substantially fill) voids between each of a superior aspect of an eyeglass frame and the face, an inferior aspect of the eyeglass frame and the face, and a nasal aspect of the eyeglass frame and the face. In some embodiments, the nasal portion of the glasses alone may form the seal between the nasal aspect of the eyeglass frame and the face, without the need for additional seal. Upon creation of the seal, the model of the seal is sent to a 3D printer to be printed and the 3D printer prints the at least the seal. In some examples, the 3D printer also prints in the eyeglass frame. The eyeglass frame and the seal may be printed together as a single part or printed separately, such that the seal can be attached to the frame after printing. In other scenarios, the frame is already printed and includes attachment components, such that when the seal is printed, it can be attached to the frame.

### BRIEF DESCRIPTION OF THE DRAWINGS

Embodiments of the present technology will be described and explained through the use of the accompanying drawings

FIG. 1 illustrates an example of various components within a system that may be used in some embodiments of the present technology.

FIG. 2 is a flowchart illustrating an example of a set of operations for creating customized eyewear in accordance with various embodiments of the present technology.

FIG. 3 is a flowchart illustrating an example of a set of operations for creating and printing customized eyewear in accordance with various embodiments of the present technology.

FIG. 4 illustrates a 3D volumetric rendering in accordance with one or more embodiments of the present technology.

FIG. 5 illustrates a depiction of a facial scan in accordance with some embodiments of the present technology.

FIG. 6 illustrates a facial scan of a bust taken with a laser scanner in accordance with various embodiments of the present technology.

FIG. 7 illustrates a conversion of a surface mesh into a 3D solid body that can be manipulated in various computer-aided design (CAD) programs in accordance with one or more embodiments of the present technology.

FIG. 8 illustrates an example of a trimmed solid body that retains crucial facial structures for glasses frame creation in accordance with some embodiments of the present technology.

FIG. 9 illustrates several examples of glasses frame converted into files that can be manipulated in various CAD programs in accordance with various embodiments of the present technology.

FIG. 10 illustrates the translation of a pair of glasses frames into frames for the treatment of dry eyes in accordance with one or more embodiments of the present technology.

FIGS. 11A-11E illustrate representations of glasses frames produced using systems and methods in accordance with some embodiments of the present technology.

FIGS. 12A-12D illustrate examples of various components of an eyewear frame that may be created in a modeling platform based on 3D imaging in accordance with one or more embodiments of the present technology.

FIG. 13 illustrates an example of glasses for the treatment of dry eyes modeled on the solid model of a face in accordance with various embodiments of the present technology.

FIGS. 14A and 14B illustrate various perspectives of glasses for the treatment of dry eyes that may be created in accordance with some embodiments of the present technology.

FIGS. 15A-15F illustrate a series of examples of aligning and orienting a facial scan in accordance with one or more embodiments of the present technology.

FIGS. 16A-16D illustrate a series of examples of aligning and orienting a pair of eyeglasses in a computer model in accordance with one or more embodiments of the present technology.

FIGS. 17A-17C illustrate an example alignment demonstration in a computer model in accordance with one or more embodiments of the present technology.

FIG. 18 is a sequence diagram illustrating an example of a set of communications between various components of a system that may be used in accordance with various embodiments of the present technology.

FIG. 19 illustrates various components of a computing system in accordance with one or more embodiments of the present technology.

The drawings have not necessarily been drawn to scale. Similarly, some components and/or operations may be separated into different blocks or combined into a single block for the purposes of discussion of some of the embodiments of the present technology. Moreover, while the technology is amenable to various modifications and alternative forms, specific embodiments have been shown by way of example in the drawings and are described in detail below. The intention, however, is not to limit the technology to the particular embodiments described. On the contrary, the technology is intended to cover all modifications, equivalents, and alternatives falling within the scope of the technology as defined by the appended claims.

### DETAILED DESCRIPTION

Various embodiments of the present technology generally relate to treatment for dry eyes. More specifically, some embodiments of the present technology relate to eyeglass frames for treatment of dry eye syndrome and corresponding three-dimensional printing systems and methods. Dry eye disease is a significant ocular problem in the United States and is estimated to affect twenty million people. Studies show that approximately fifteen percent of elderly people in the United States are affected by dry eye disease and as many as thirty-three percent suffer from dry eye disease in Asia. Twenty-five percent of patients visiting ophthalmic practices report symptoms of dry eye disease. Incidences of dry eye disease increases at higher altitudes, making the problem more prevalent and significant in especially dry places at high elevations.

Dry eye disease is most often associated with advancing age, female sex, low relative humidity, use of certain medications, and cigarette smoking. Environmental issues greatly affect relative humidity, including the relative humidity of certain areas of the world and internal environments such as office buildings, homes, and automobiles. The internal relative humidity of many office buildings and vehicles is often kept at extremely low levels. Moreover, dry eye disease has a significant impact on a person's quality of life. Patients with dry eye disease often experience significant ocular discomfort as they try to complete visual tasks. Almost all treatment strategies for dry eye disease are directed at medical therapies. Treatment often starts with artificial tears, and may advance to the use of punctal plugs, cyclosporin drops, or autologous serum tears. In recent years, more novel therapies such as LipiFlow Vectored Thermal Pulsation Therapy have been developed aiming to improve Meibomian gland function. Traditional eyewear solutions for dry eyes are not custom fit to the shape of a person's face. Since the human face is quite variable in shape, the fit of generic dry eyeglasses is often quite poor. Consequently, the treatment of dry eye disease is also poor. Current dry eyeglasses attempt to seal the entire orbit around the eye.

In contrast to these traditional eyewear solutions, various embodiments of the present technology allow the frame's lateral aspect to not be sealed off in order to achieve higher humidity levels. For example, in some embodiments, the frame's lateral aspect of the present technology is not sealed off while still achieving humidity levels that are higher than the humidity levels of the surrounding environment. For example, depending on the external humidity level and the size and shape of the open portions (e.g., on the temporal aspects of the frame), various embodiments may see an internal humidity level range from below ten percent to over ninety-five percent. Moreover, the internal humidity level may vary during the day, based on the location of the individual (e.g., outside vs. inside an office building), perspiration of the individual, materials of the seal and/or frames, placement of the eyewear on the individual's face, and/or other factors. For example, various embodiments may keep the humidity level between twenty percent and sixty percent, thirty percent and eight percent, five percent and twenty percent, five percent and eighty percent, sixty percent and one hundred percent, and/or many other ranges depending on the design, external conditions, individual traits (e.g., perspiration, tear production, etc.).

By not sealing the lateral aspect of the glasses, a more traditional appearance is achieved, eliminating a wearer's need to feel uncomfortable with the appearance of their therapeutic glasses. In addition, various embodiments of the present technology can include an encompassing processing through which a normal appearing eyeglass frame can be custom built and automatically printed for the use of evaporative dry eye relief. Prior solutions to dry eye syndrome have failed to achieve an encompassing process through which a custom pair of eyeglass frames are built and able to raise the humidity around the cornea while retaining aesthetic appeal.

In some embodiments of the present technology, computer aided design (CAD) techniques and accompanying user interfaces are provided that are capable of utilizing a multi-dimensional facial scan file of any individual's face. With the use of various existing computer CAD software and a database of .dxf files (e.g., containing the outline of the desired frames), automatically 3D prints a unique pair of glasses that conforms to the facial anatomy and topology of that individual's face. These custom fit frames form a partial seal around the orbit which traps water vapor, significantly raising the relative humidity over the eye. Various embodiments of the present technology can form a seal nasally, superiorly, and inferiorly, but the temporal area is purposely left somewhat open to allow for limited air circulation. This limited air circulation prevents the eyeglasses from fogging during normal activities and allows for a normal appearance of the frames for improved aesthetics.

In some embodiments a method of producing eyewear for the treatment of dry eyes includes acquiring a scan (e.g., using a three-dimensional (3D) scanner) of at least a portion of a face of a user. The scan of the face can then be converted into a 3D computer model and a computer model of an eyewear apparatus can then be generated. In order to use the facial scan in a computer model, some embodiments may use a computer program or platform to orient the scans in such a manner that it may be used by CAD software to create eyeglass frames. Such platforms or programs include, but are not limited to, Geomagic Wrap, Geomagic Freeform, VR&D GENESIS, Dassault Systèmes SolidWorks Composer, Siemens STAR-CCM+, VectorEngineer, ANSYS Meshing, ANSYS DesignXplorer, ANSYS SPEOS, and VXelements.

In various embodiments, the eyewear apparatus may include a frame having a front composed of two eyepieces to hold custom lenses. A ridge-like interface on the rear periphery of the front of the frame can be designed to engage (e.g., seal) a large portion of the front of the frame with the face of the user. For example, the ridge-like structure can be designed such that a portion of the periphery is engaged with the skin on normal wearing. The amount of the ridge-like structure engaging the skin only need to be sufficient to increase the humidity levels for the individual to a level that will provide therapeutic relief. Note that different individuals may find relief at different humidity levels.

Depending on the embodiment, the engagement of the ridge-like structure with the skin may be greater than 50% but less than 90%, more than 90%, more than 40%, or the like. The particular level of engagement may be selected based on individual characteristics of the individual that will be wearing the frames (e.g., activity level, office worker, levels of tear production, sweat production, etc.) and/or external factors (e.g., geographic location, external humidity levels, frame materials selected, etc.).

For example, the superior portion, an inferior portion, and a nasal portion of the front of the frame (or a substantially large amount of each portion thereof) and the 3D computer model of the face. In some embodiments, the computer model of the eyewear apparatus can be converted into a pair of eyeglasses via a 3D printer (e.g., locally or remotely). In some embodiments, converting the scan of the at least a portion of the face in a 3D model of the face can include generating a surface mesh of at least a portion of the face based on the scan, converting the surface mesh into a solid body model, and importing the solid body model into a 3D computer model of the face.

Various embodiments of the present technology provide for a wide range of technical effects, advantages, and/or improvements to computing systems and components. For example, various embodiments include one or more of the following technical effects, advantages, and/or improvements: 1) integrated use of facial scans, computer-aided-design, and 3D printing to create custom fitting eyewear to reduce the systems of dry eyes; 2) integrated use of design techniques to create eyewear (e.g., frames) with a custom fit to the shape of a person's face; 3) remove the need for water reservoirs; 4) use of an incomplete seal (e.g., an opening in the frame's lateral or temporal aspect) around the orbits of the eyes while still achieving sufficiently high humidity levels in the approximate ranges of forty to ninety percent; 5) achieves substantial improvement in humidity without compromising aesthetics; 6) use of non-routine computer operations to create an encompassing process through which a normal appearing eyeglass frame can be custom built and automatically printed for the use of evaporative dry eye relief; and/or 7) changing the manner in which a computing system reacts to user interactions and feedback.

In the following description, for the purposes of explanation, numerous specific details are set forth in order to provide a thorough understanding of embodiments of the present technology. It will be apparent, however, to one skilled in the art that embodiments of the present technology may be practiced without some of these specific details. The techniques introduced here can be embodied as special-purpose hardware (e.g., circuitry), as programmable circuitry appropriately programmed with software and/or firmware, or as a combination of special-purpose and programmable circuitry. Hence, embodiments may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions.

The phrases “in some embodiments,” “according to some embodiments,” “in the embodiments shown,” “in other embodiments,” and the like generally mean the particular feature, structure, or characteristic following the phrase is included in at least one implementation of the present technology, and may be included in more than one implementation. In addition, such phrases do not necessarily refer to the same embodiments or different embodiments.

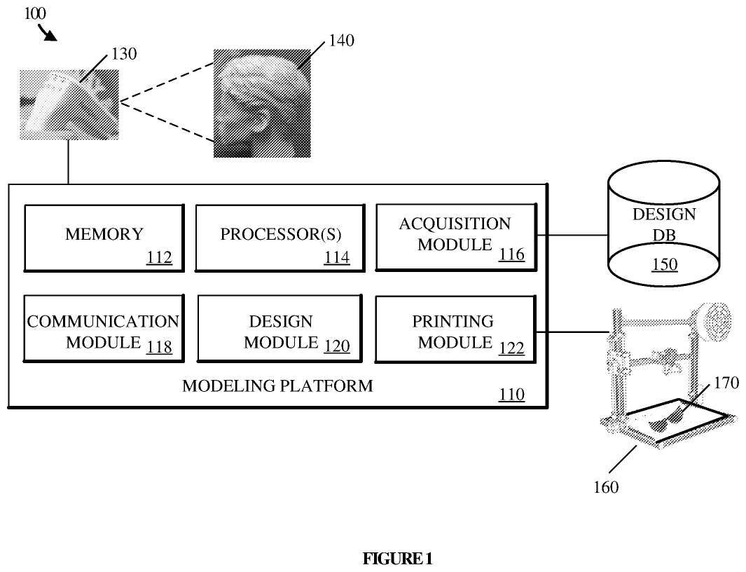

FIG. 1 illustrates an example of various components within a system 100 that may be used in some embodiments of the present technology. As illustrated in FIG. 1, the system 100 can include modeling platform 110, scanner 130 to scan individual 140, a design database 150, and a 3D printer 160. In accordance with various embodiments, system 100 can be designed to create aesthetically pleasing frames while producing humidity levels high enough to adequately lessen evaporative dry eye. Some embodiments of modeling platform 110 include various techniques with accompanying user interfaces that are capable of utilizing a multi-dimensional facial scan file (e.g., created using scanner 130) of an individual's face 140 and, with the use of various computer software and databases, automatically three-dimensional (3D) print a unique pair of glasses that conform to the facial anatomy and topography or structure of the individual's face.

In the embodiments illustrated in FIG. 1, modeling platform 110 can include memory 112, processor(s) 114, acquisition module 116, communication module 118, design module 120, and printing module 122. Each of these modules in modeling platform 110 can be embodied as special-purpose hardware (e.g., one or more ASICS, PLDs, FPGAs, or the like), or as programmable circuitry (e.g., one or more microprocessors, microcontrollers, or the like) appropriately programmed with software and/or firmware, or as a combination of special purpose hardware and programmable circuitry. Other embodiments of the present technology may include some, all, or none of these modules and components along with other modules, applications, and/or components. Still yet, some embodiments may incorporate two or more of these modules and components into a single module and/or associate a portion of the functionality of one or more of these modules with a different module. For example, in one embodiment, acquisition module 116 and design module 120 can be combined into a single module for creating customized eyewear for dry eyes.

Memory 112 can be any device, mechanism, or populated data structure used for storing information. In accordance with some embodiments of the present technology, memory 112 can encompass any type of, but is not limited to, volatile memory, nonvolatile memory and dynamic memory. For example, memory 112 can be random access memory, memory storage devices, optical memory devices, media magnetic media, floppy disks, magnetic tapes, hard drives, SDRAM, RDRAM, DDR RAM, erasable programmable read-only memories (EPROMs), electrically erasable programmable read-only memories (EEPROMs), compact disks, DVDs, and/or the like. In accordance with some embodiments, memory 112 may include one or more disk drives, flash drives, one or more databases, one or more tables, one or more files, local cache memories, processor cache memories, relational databases, flat databases, and/or the like. In addition, those of ordinary skill in the art will appreciate many additional devices and techniques for storing information which can be used as memory 112.

Memory 112 may be used to store instructions for running one or more applications or modules on processor(s) 114. For example, memory 112 could be used in one or more embodiments to house all or some of the instructions needed to execute the functionality of acquisition module 116, communication module 118, design module 120, and/or printing module 122. Some embodiments of modeling platform 110 may include an operating system which provides a software package that is capable of managing the hardware resources of modeling platform 110. The operating system can also provide common services for software applications running on processor(s) 114.

Acquisition module 116, in some embodiments, can be used to control scanner 130 to collect a facial scan of individual 140. In other embodiments, the facial scan may be collected independently and transmitted to the modeling platform. Some embodiments of the present technology may use scanning-based systems, imaging systems, and/or other systems that can applanate against the face and measure contours to create a digital surface model. Communication module 118 can send and receive communication with other components of the system (e.g., scanner 130, design database 150, 3D printer 160, user devices, etc.). Design database 150 may be locally accessible or remotely accessible via a cloud or similar platform.

Design module 120 can initiate a design workflow based on the scan acquisition in order to create custom fit eyewear/frames. In accordance with various embodiments, these custom fit frames form a partial seal around the orbital sockets in order to trap water vapor between the lenses and the eyes of individual 140 thereby significantly raising the relative humidity over the eye. The partial seal formed around the orbital sockets may vary depending on several individual and external factors. For example, the partial seal may be less than 90%, less than 80%, less than 70%, less than 60%, less than 50%, etc.). In some embodiments the partial seal may indicate a complete seal along a majority of the perimeter of the back side of the front of the frame.

For example, some embodiments of the frames can include a ridge relief that is integrated into the frame or separately attached. The ridge relief can follow the curvatures of the individual's face and sufficiently engage the individual's face during normal wear to form a seal to raise the humidity level around the eye (e.g., by trapping evaporation from the skin, tears, etc.) relative to the external environmental humidity level. In some embodiments, the seal is around a majority of the individual orbital sockets and may form a complete or substantial seal (e.g., greater than 60%-70%) nasally, superiorly, and inferiorly. The substantial seal generally refers to the creation of enough contact between the ridge relief to create an increase in humidity level relative to a typical environment that individual will be located in. As such, the amount of engagement with the skin may vary in different amounts depending on a number of factors (e.g., individual, frame design, seal designs, needs for therapeutic relief, external conditions, activity level, and the like) For example, the engagement may be greater than 50% but less than 90%, more than 90%, more than 40%, or the like. In some embodiments, the contacting portion may be contiguous or may include patterns of engagement and openings.

Some embodiments may intentionally leave the temporal sides open (e.g., fail to engage the skin of the individual's face) to allow for limited air circulation and therefore increased humidity. The limited air circulation enabled by the present design prevents the eyeglasses from fogging during normal activities while also creating a traditional eyeglass appearance of the frames for improved aesthetics.

Printing module 122 can translate the design to a file which can be transmitted (e.g., using communication module 118) to 3D printer 160 which can then print custom frames 170 and/or ridge relief that can be attached to custom or off-the-shelf frames. In some embodiments, 3D printer 160 can include multiple materials allowing different parts of the frames to be printed with different materials (e.g., Acrylonitrile Butadiene Styrene (ABS), Acrylonitrile Styrene Acrylate (ASA), carbon fiber filaments, High Impact Polystyrene (HIPS), Nylon, PolyPropylene (PP), Plasticized Copolyamide (PCTPE), Polycarbonate, Polypropylene, Polyethylene Terephthalate (PET), Polyethylene Terephthalate Glycol (PETG), Polylactic Acid (PLA), Polyamide With Chopped Glass Fiber Strands, Thermoplastic Polyurethane (TPE), Thermoplastic Polyurethane (TPU), etc.). For example, the ridge relief may be printed with a softer material (e.g., a TPE filament such as PRO Series Flex that feels and acts much like flexible rubber, soft PLA filament, etc.) that can be compressed when engaged with the individual's skin.

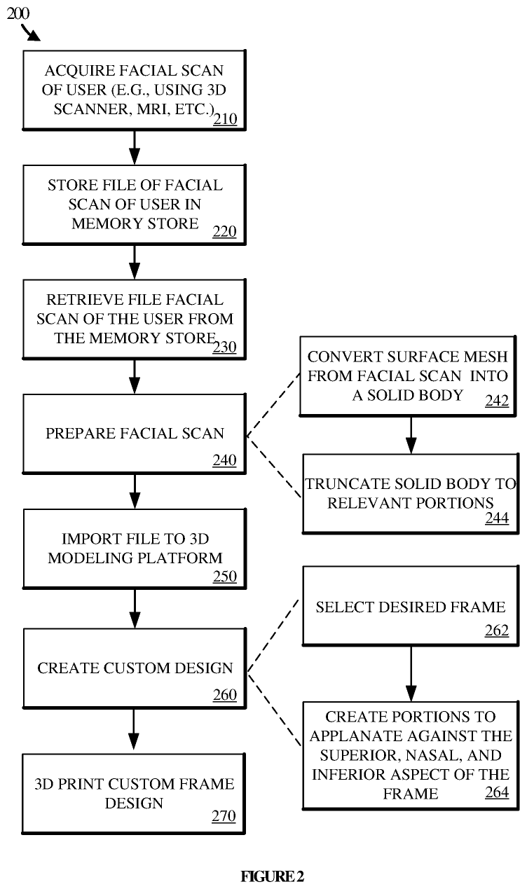

FIG. 2 is a flowchart illustrating an example of a set of operations 200 for creating customized eyewear in accordance with various embodiments of the present technology. As illustrated in the embodiments shown in FIG. 2, acquisition operation 210 can acquire a facial scan of a user (e.g., using a handheld 3D scanner, MRI machine, etc.). In accordance with various embodiments, the facial scan acquisition may be requested by the modeling platform or may be collected independently (e.g., for another purpose). For example, there are numerous ways in which an individual's three-dimensional facial scan could be acquired. Examples include, but are not limited to, contact 3D scanners, modulated light 3D scanners, volumetric 3D scanners, stereoscopic and photometric 3D scanners, CT, MRI, and similar scanning methods. Regardless of the source of the individual's facial topology and contours, the scan can be used to easily and accurately produce custom frames that precisely fit an individual's face.

Once a facial scan is successfully acquired, storing operation 220 allows the file of the facial scan to be stored in a memory store (e.g., database, local hard drive, etc.). Using retrieval operation 230, the modeling platform may find and access any files produced from the facial scan (e.g., in a cloud or universal database, locally on a server or individual computer, or a similar storage location). The files may be in a 3D format including but not limited to STL, OBJ, FBX, COLLADA, 3DS, IGES, STEP, VRML/X3D, MSH, MESH, C4D, RAW, BLEND, PKY, or the like. Additionally, formats such as a DICOM file, which encodes the 3D information contained in CT and MRI scans, could be secondarily converted into one of the aforementioned file types and then used for frame production.

After accessing a usable file, preparation operation 240 can prepare the file and data for generation of a solid model preferred for frame creation. For example, the file can be imported into a program that can take a surface mesh file and convert it into a solid body using conversion operation 242. The solid body may be used more efficiently for eyeglass frame production. In some embodiments, the present step may not be necessary if the file type does not require the conversion. Some standalone programs can be used to perform the conversion of a surface mesh into a solid body. Other 3D computer aided design (CAD) platforms have the native capability to take the various file formats and convert them into solid bodies or boundary representations which can ultimately be utilized during eyeglass frame generation.

Once the proper representation (e.g., file format) of the individual's original facial scan is produced, the system may use truncation operation 244 to truncate the original scan into a smaller solid body that is easier to manipulate within various 3D CAD software programs. For example, if the system processing the 3D file is not powerful enough to manipulate the original solid body, the dimensions can be reduced, manually or automatically, or the original solid body in order to produce a smaller file size that makes it easier to manipulate across all types of hardware. The reduced file size may be accomplished by trimming the original solid body into a solid body that only includes the crucial features for creating the eyewear, rather than the entire head. Crucial features of the facial structure may include nose, eyes, forehead, ears, and other related structures.

Another method of reducing the overall file size may be to reduce the file quality of the original solid body (i.e., compress) by reducing the number of polygons within the solid body. However, compressing the file may compromise fine details of the solid body's surface, but may ultimately leave the overall facial topology unaltered, which is the most crucial parameter necessary for proper implementation.

After preparing the solid body, importation operation 250 can be used to import the file into a dedicated 3D modeling platform in accordance with the present technology. The file, either the original solid body or the truncated file, can be imported into a large variety of robust 3D modeling platforms including but not limited to 3D slash, LibreCAD, Photoshop CC, SculptGL, SelfCAD, TinkerCAD, Clara.io, DesignSpark, FreeCAD, Meshmixer, Moment of Inspiration (MoI), nanoCAD, OpenSCAD, Sculptris, SketchUp, 3ds Max, AutoCAD, Blender, Cinema 4D, modo, Mudbox, Onshape, Poser, Rhino3D, ZBrush, CATIA, Autodesk Fusion 360, Inventor, SolidWorks, and many other platforms not mentioned here for the sake of brevity.

Once imported into a 3D modeling platform, the system may utilize various creation operations 260 and native functions (i.e., “SKETCH”, “CREATE”, “MODIFY”, “ASSEMBLE”, “CONSTRUCT”, “INSPECT”, “INSERT”, “MAKE”, “ADD-INS”, “SELECT”, etc.), including the functions contained within each of these broader function categories, to create the individualized glasses.

In some embodiments, many existing glasses frame style can be selected during selection operation 262 and imported into any of the aforementioned 3D modeling programs. The frame can be imported into the platform in addition to the solid body, in order to custom tailor the desired frame style to an individual's face for the most ideal fit. The frame styles, in some embodiments, are newly generated, or contained in a database containing DXF files that represent the outlines of various frame styles of current or future glasses. The database may be locally accessible or remotely accessible via a cloud or similar platform. Programs that can create, open, and edit DXF files could be utilized to create the database that would provide a library of available styles to the wearer. Programs for this function include but are not limited to AutoCAD, CorelCAD, Serif DrawPlus, Autodesk Design Review, Autodesk DWG TrueView, Dassault Systemes SolidWorks eDrawings Viewer, LibreCAD, and the like. These programs may be utilized to create the DXF outlines, which may then be accessible to the algorithm, allowing the wearer to choose any style of glasses they desire.

Once the individual's facial scan, represented by the original or truncated solid body, and the DXF file, representing the customer's desired style of frames, have both been imported into the 3D modeling platform, the native functions of the respective platform can be utilized, manually or automatically, to produce the glasses frames that match the topology of the individual's face. In creation operation 264, the automated design will create portions of the frames that will applanate against the superior, nasal, and inferior aspect of the frames, creating a seal with the skin of the face to trap humidity. The temporal aspect will be intentionally left unsealed, creating a similar effect to traditional eyeglass frames. The open temporal aspect allows for a normal eyeglass appearance while allowing the system to trap humidity to levels that produce relief from evaporative dry eye.

The process for creating the frames can be achieved using several techniques. However, various embodiments may automatically produce a file that can be uploaded to a 3D printer and printed using printing operation 270. File formats that can be uploaded to the 3D printer in accordance with the present technology include but are not limited to OBJ, STL, and similar file formats capable of 3D printing. Thus, any style of frames can be printed, in a large variety of colors and a large variety of 3D printable materials, for the wearer.

The 3D file produced in the processes described above may then be saved as a file (locally, universally, on a cloud, on a server, etc.) that can be recognized and printed by a 3D printer in any material reasonable for producing functional and durable eyeglass frames. In some scenarios, the eyeglass frames ultimately produced may be subject to additional processing (removal of 3D printing support structures, sanding, coating, lens placement, and the like) and the processes may be accomplished in several different ways. The need for further processing can be determined in each situation based on material, style, coating, and lenses used for each individual pair of frames.

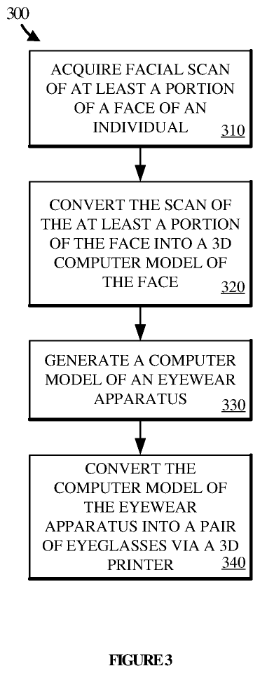

FIG. 3 is a flowchart illustrating an example of a set of operations, operations 300, for creating customized eyewear in accordance with various embodiments of the present technology. As illustrated, facial scan operation 310 acquires a facial scan of at least a portion of a user's face (e.g., using a handheld 3D scanner, MRI machine, etc.). In accordance with various embodiments, there are numerous ways in which an individual's three-dimensional facial scan could be acquired. Acquisition examples include, but are not limited to, contact 3D scanners, modulated light 3D scanners, volumetric 3D scanners, stereoscopic and photometric 3D scanners, CT, MRI, and similar scanning methods. Regardless of the source of the individual's facial topology and contour data, the scan may be used to easily and accurately produce custom frames that precisely fit at least a portion of the individual's face.

The next step is conversion operation 320, where the facial scan is converted into a 3D computer model of the face. The facial scan may be exported to a program that can take the file of the scan and convert it into a solid body. The solid body may be used more efficiently for eyeglass frame production. Some standalone programs can be used to perform the conversion of a surface mesh into a solid body. Other 3D computer aided design (CAD) platforms have the native capability to take the various file formats and convert them into solid bodies or boundary representations which can ultimately be utilized during eyeglass frame generation.

In the next step, generation operation 330, a computer program or the like may take the converted file and generate a computer model of the eyewear apparatus that fits the measurements and topology of the portion of the face captured. The eyewear apparatus may comprise a frame of the eyeglasses, a seal between a superior portion of the frame and the three-dimensional computer model of the face, a seal between an inferior portion of the frame and the three-dimensional computer model of the face, and a seal between a nasal portion of the frame and the three-dimensional computer model of the face. At printing operation 340, the model of the eyewear apparatus is sent to a 3D printer which converts the model into a pair of eyeglasses custom-fit to the scanned portion of the individual's face.

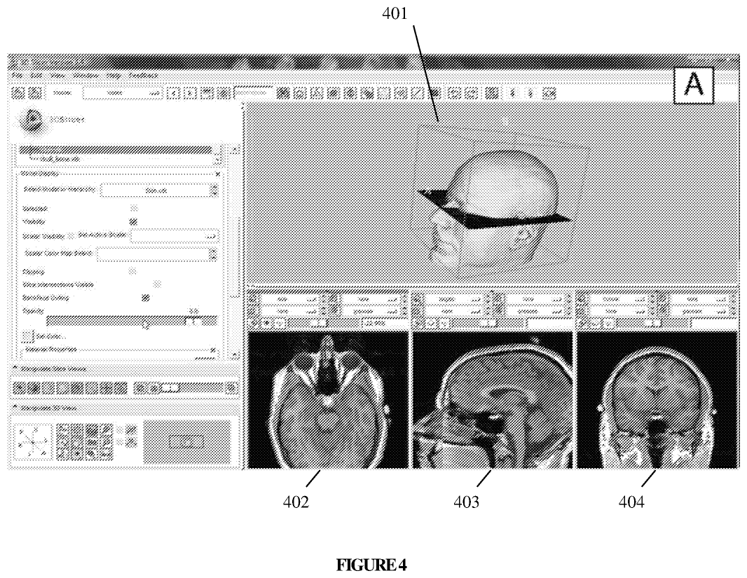

FIG. 4 illustrates an example of a 3D volumetric rendering 401 of sequential 2D MRI slices 402, 403, and 404 using a 3D slicer platform in accordance with some embodiments of the present technology. 2D MRI slice 402 demonstrates a top-view of the 3D volumetric rendering 401 while 2D MRI slice 403 shows a lateral-view, and 2D MRI slice 404 illustrates a rear-view. In some embodiments, measurements may be taken from 3D volumetric rendering 401 for frame production of the present disclosure.

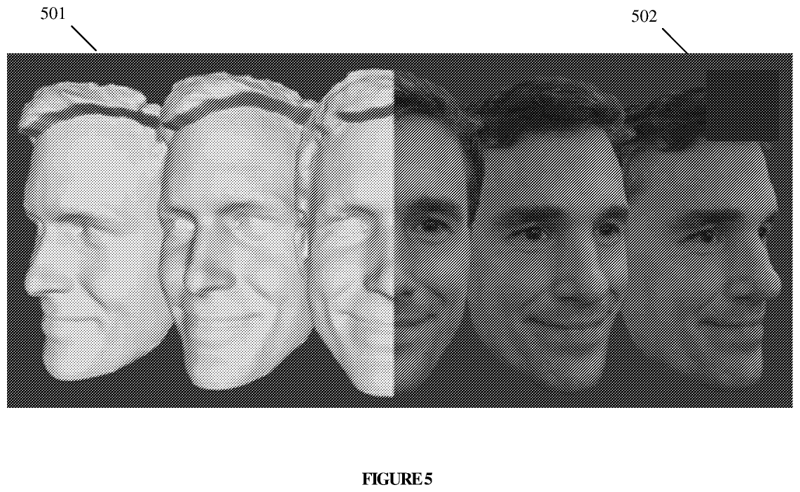

FIG. 5 illustrates an example of a 3D rendering of a surface mesh model created from a photometric 3D scanner. In some embodiments, the photometric 3D scanner provides data on a user's facial topology to be used for frame production in the frame modeling systems. A scan of the user demonstrates user image 502 translating into photometric scan 501. Photometric scan 501 produces measurements consistent with the topology of the user being scanned.

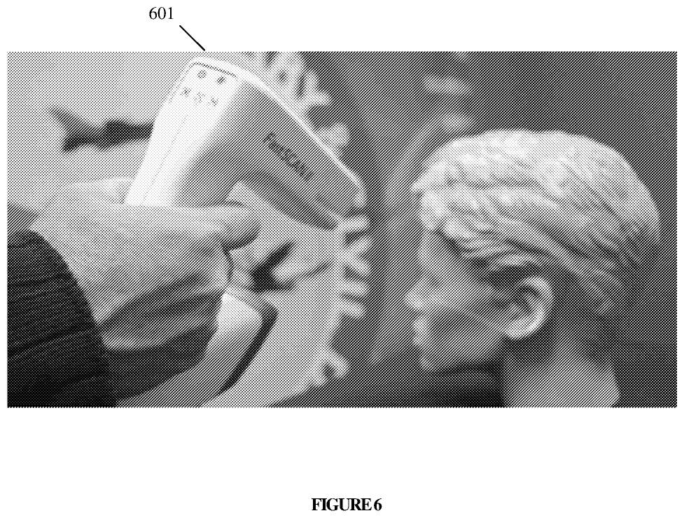

FIG. 6 illustrates an example of a facial scan being taken by a handheld 3D laser scanner 601. There are numerous ways in which an individual's three-dimensional facial scan could be acquired including, but not limited to, contact 3D scanners, MRI (see, e.g., FIG. 4), CT, stereoscopic and photometric 3D scanners (see, e.g., FIG. 4), handheld laser 3D scanners (see, e.g., FIG. 6), modulated light 3D scanners, and volumetric 3D scanners. Regardless of the source of the individual's facial topology, custom frames for the treatment of dry eyes can be accurately produced to exactly fit an individual's face using the facial and eyeglass frame modeling systems disclosed herein. As previously discussed, once a facial scan is successfully acquired, various embodiments of the present system may access the file of the 3D facial scan, which may be in one of a variety of 3D file formats.

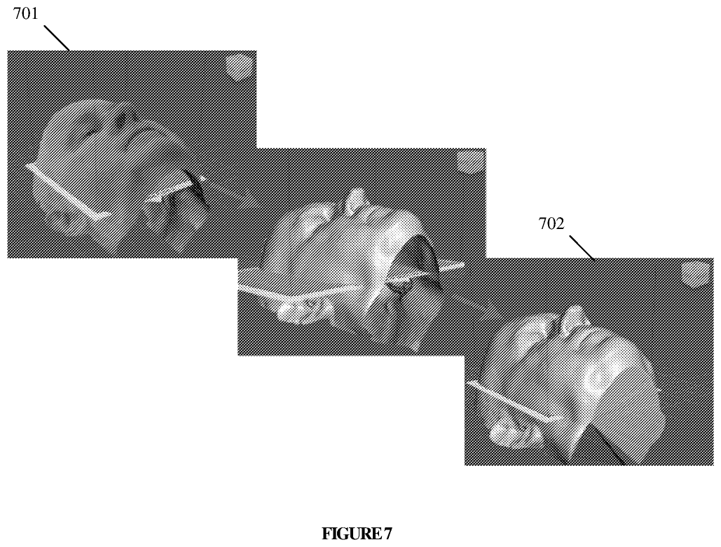

FIG. 7 illustrates a conversion of a surface mesh into a 3D solid body that can be manipulated in various computer-aided design (CAD) programs in accordance with one or more embodiments of the present technology. To begin, initial scan step 701 includes taking an image of an individual's head to be input into a CAD program to measure the contours of the individual for data processing. At scan conversion completion step 702, the individual's head scan has been converted into 3D solid body scan that can be used in various programs to determine relevant measurements for frame production of the present disclosure. As mentioned, the 3D scan of the individual may be in one of a variety of 3D file formats to be input in a CAD program.

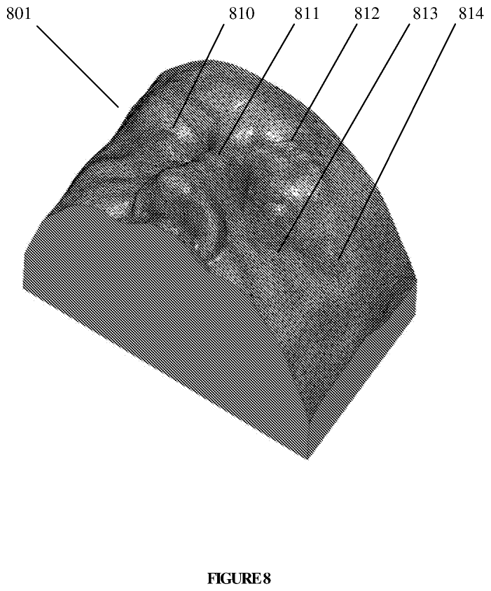

FIG. 8 illustrates an example of a trimmed solid body that retains crucial facial structures for glasses frame creation in accordance with some embodiments of the present technology. Crucial facial structure 801 displays the critical measurement areas that may be obtained using one of the aforementioned scanning processes to produce the frames in the present disclosure. Topological measurements may be taken from various areas around eye 810, including, but not limited to, nasal structure 811, eyebrow structure 812, under-eye structure 813, and temporal structure 814. Various measurements such as these can be used to easily and accurately produce custom frames that precisely fit an individual's face.

Some embodiments of the present technology allow for the ability to select and import several existing glasses frame styles into any of the aforementioned 3D modeling platforms, in conjunction with the individual's facial scan, in order to custom-tailor the desired frame style onto the individual's face for the most ideal fit. These frame styles would be contained in a database (e.g., design database 150 in FIG. 1), either locally accessible or accessible via the cloud, containing DXF files that represent the outlines of various frame styles of current or future glasses.

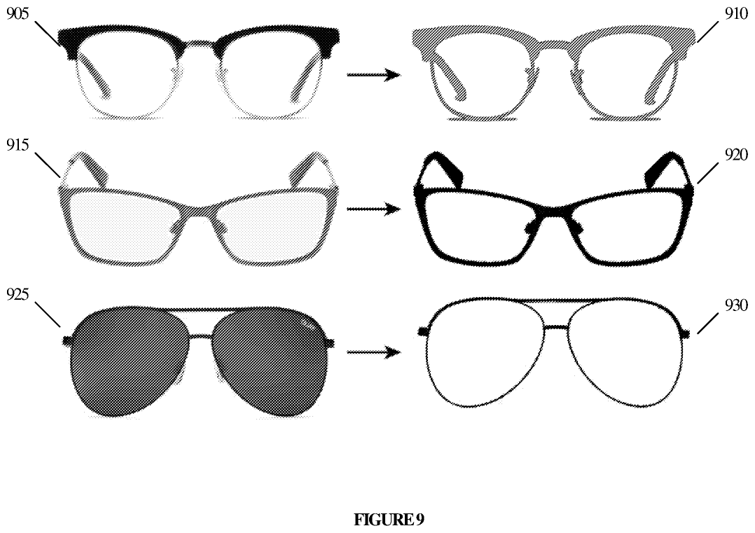

FIG. 9 illustrates several examples of glasses frames 905, 910, 915, 920, 925, and 930 that can be converted into files to be manipulated in various CAD programs in accordance with various embodiments of the present technology. Programs that can create/open/edit DXF files could be utilized to create this database that would provide a library of available styles available to the algorithm and, therefore, the consumer. Programs including, but not limited to, AutoCAD, CorelCAD, Serif DrawPlus, Autodesk Design Review, Autodesk DWG TrueView, Dassault Systemes SolidWorks eDrawings Viewer, LibreCAD, etc. could be utilized to create these DXF outlines, which would then be accessible to the algorithm, allowing the customer to choose any style of glasses they desire.

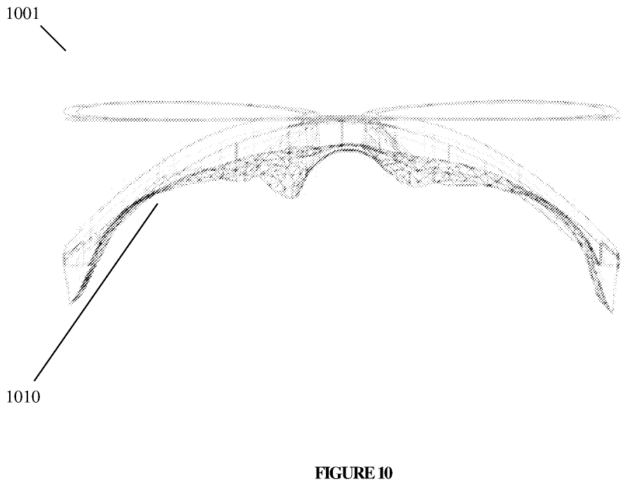

FIG. 10 illustrates the translation of a pair of glasses frames into frames 1001 for the treatment of dry eyes in accordance with one or more embodiments of the present technology. Frame curvature 1010 resembles a facial topology of an individual measured from one or more facial scans acquired using various methods, such as 3D volumetric or photometric scans or the like. Frame curvature 1010 may be used as input measurements when printing a 3D model of the frames 1001 to provide a custom fit of eyeglasses for the wearer of the frames 1001 for its intended purpose.

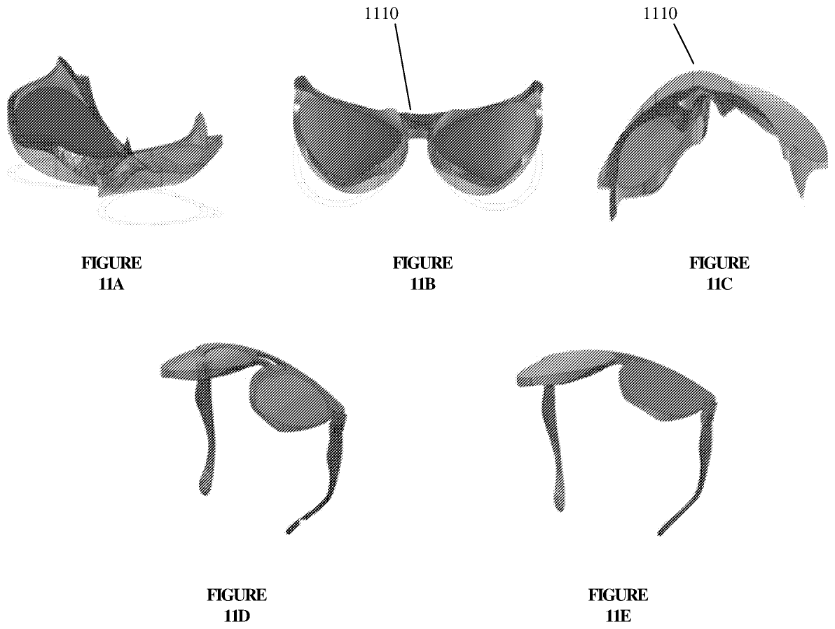

FIGS. 11A-11E illustrate representations of a pair of glasses frame produced using systems and methods in accordance with some embodiments of the present technology. Each of FIGS. 11A-11E demonstrates the curved, custom fit to a user's scanned head/face using one or more facial scanning techniques such as 3D volumetric rendering, 3D photometric scanning, or the like. The frame curvature 1110 depicted in FIGS. 11B and 11C exemplifies a custom curvature formed to fit the individual's face captured in the aforementioned facial scans.

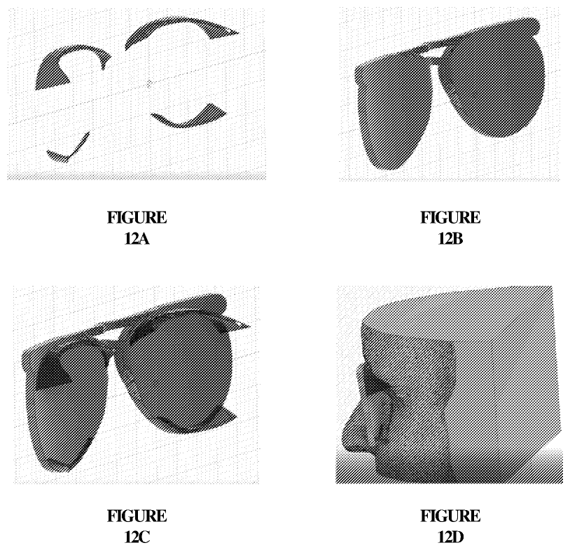

FIGS. 12A-12D illustrate examples of various components of an eyewear frame that may be created in a modeling platform based on 3D imaging in accordance with one or more embodiments of the present technology. For example, in accordance with some embodiments, once the individual's facial scan (represented by the original or truncated solid body) and the DXF file (representing the customer's desired style of frames) have both been imported into the 3D modeling platform, the algorithm would then utilize all of the native functions of the respective platform (for example functions found in Autodesk Fusion 360: “SKETCH”, “CREATE”, “MODIFY”, “ASSEMBLE”, “CONSTRUCT”, “INSPECT”, “INSERT”, “MAKE”, “ADD-INS”, “SELECT”, etc. [including the functions contained within each of these broader categories]) in order to produce the glasses frame that matches the topology of the individual's face. As illustrated, FIG. 12A demonstrates truncated curvature pieces of a pair of eyeglasses that, added to the pair of eyeglasses shown in FIG. 12B, create custom-fit eyeglasses shown in FIG. 12C. Using the individual's facial scan and the custom-fit eyeglasses, FIG. 12D displays a tight, nearly sealed pair of eyeglasses that fit the exact topology of the individual.

This automated design will create portions of the frames that will applanate against the superior, nasal, and inferior aspect of the frames, creating a seal with the skin of the face to trap humidity. The temporal aspect will purposefully be left open as is seen with normal eyeglass frames. This will allow for a more normal appearance of the eyeglass frames while still allowing the entire system to trap humidity to levels that produce relief from evaporative dry eye. The exact process for creating this file can be accomplished using several methods and are certainly subject to change, however, any sequence, on any 3D modeling platform, the algorithm would automatically produce a file (either OBJ, STL, or any other file format capable of 3D printing) that would have the ability to be uploaded to a 3D printer and print any desired style of frames, in any color, and any 3D-printable material (within reason) for the customer.

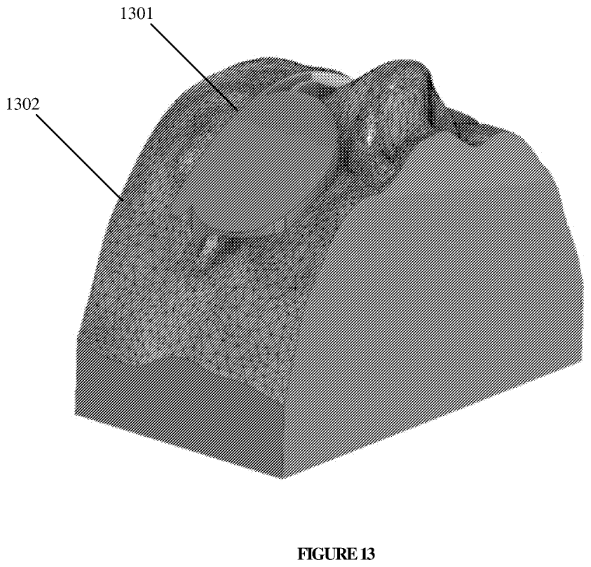

FIG. 13 illustrates an example of custom fit glasses for the treatment of dry eyes 1301 modeled on the solid model of a face 1302 in accordance with various embodiments of the present technology. Solid model of a face 1302 may be obtained using one or more of the various aforementioned processes. Additionally, the custom fit glasses for the treatment of dry eyes 1301 may be created using data from the solid model of a face 1302 also using one or more of the various aforementioned processes.

The 3D file may then be saved as a file (either locally on a personal device or universally on a cloud or server using design database 150 from FIG. 1, for example) that can be recognized and printed by a 3D printer in any material that can reasonably be used to produce functional and durable eyeglass frames. The eyeglass frames that are ultimately produced may need some additional processing (e.g., removal of 3D printing support structures, sanding, coating, lens placement, etc.) and these processes could be accomplished in several different ways but can be determined in each situation based on the material, style, coating, and lens used for each individual pair of frames.

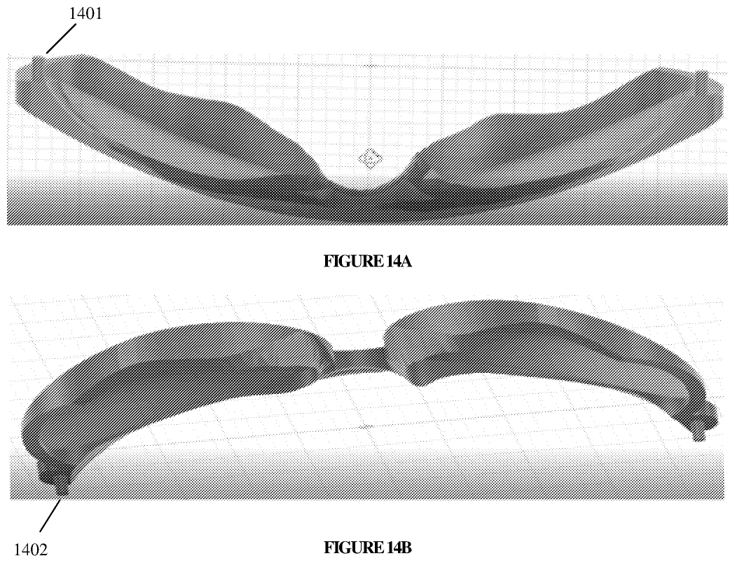

FIGS. 14A and 14B illustrate various perspectives of glasses for the treatment of dry eyes that may be created in accordance with some embodiments of the present technology. As illustrated in FIGS. 14A and 14B, the frames may include post 1401 and post 1402, respectively, on the interior of the front portion of the frames which can be used to press fit additional seals or inserts allowing the frames to follow the contour of the facial features of the individual.

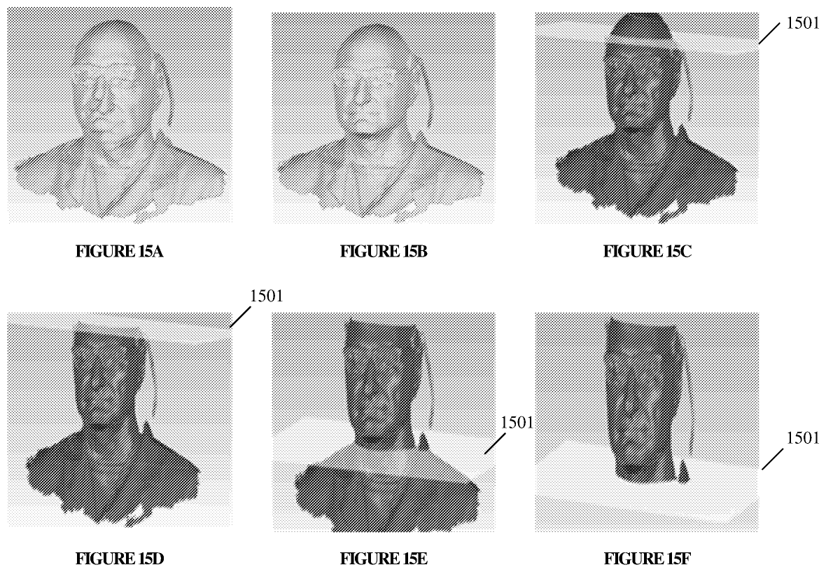

FIGS. 15A-15F illustrate a series of examples of aligning and orienting a facial scan in accordance with one or more embodiments of the present technology. The figures depicted here demonstrate a process using multiple facial scans to produce the desired result. In one facial scan, the individual is wearing a pair of eyeglasses, and in another, the individual is not wearing the eyeglasses. After the facial scans of the individual is captured, a computer model is used to aggregate the scans.

As shown in FIG. 15A, the multiple scans captured may be overlaid in a software program. The program instructions auto-align or orient the scans properly in relation to each other such that the renderings of the individual match up, as depicted in FIG. 15B. This auto-alignment process shown in FIGS. 15A and 15B prepares the scan to be exported into a CAD model. Before exporting, the software program may remove any extraneous data points to increase the reliability of the auto-alignment process and create a best-fit for the multiple facial scans.

In some embodiments, layer scanner 1501, as shown in FIG. 15C, may scan a data point cloud resembling the aligned facial scans. Layer scanner 1501 can identify and delete one or more extraneous data points. For example, in FIG. 15C, layer scanner 1501 begins at the top of the individual's head and deletes unnecessary data points. The result of this removal can be seen in FIG. 15D. Next, layer scanner 1501 continues scan the face in FIG. 15E, and it removes data points below the individual's head at the final step of the process in FIG. 15F. This process decreases the number of data points that a software program has to use to calculate a best-fit model between the multiple facial scans acquired. Moreover, the removal of extraneous data points can decrease file size and the number of points in the point cloud without altering the overall structure of the facial scan, increasing the reliability of the auto-alignment process.

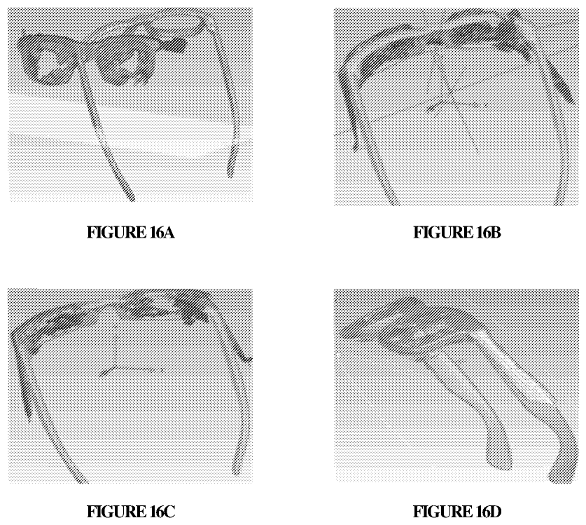

FIGS. 16A-16D illustrate a series of examples of aligning and orienting a pair of eyeglasses in a computer model in accordance with one or more embodiments of the present technology. The Figures depicted here demonstrate a process of aligning multiple scans comprising a scan of a pair of eyeglasses and a scan of an individual wearing the eyeglasses in a desired position. FIG. 16A illustrates out-of-alignment scans. Using an auto-alignment process, a program may be able to match up the eyeglasses to the position in which the individual was wearing them, as shown in FIGS. 16B and 16C. Moving to FIG. 16D, the program may perform data refinement to remove extraneous data points and prepare the rendering for export into a CAD program. After data refinement, the potential space that exists between the posterior surface of the glasses and individual's face may be used to determine the space to be filled in order to generate a seal between the eyeglass frames and the face in order to retain moisture and increase the relative humidity in accordance with one or more embodiments of the present disclosure.

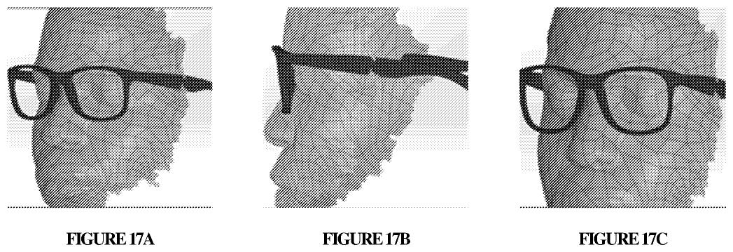

FIGS. 17A-17C illustrate an example alignment demonstration in a computer model in accordance with one or more embodiments of the present technology. Once one or more facial scans are captured and aligned, the facial scans are imported into a CAD program. In some embodiments, the facial scans may be formatted in a file type capable of being imported into a CAD program. Such file formats that may be supported by the CAD program include, but are not limited to, .STL (Stereolithography), .OBJ (Object File), .IGES (Initial Graphics Exchange Specification), .STEP (Standard for the Exchange of Product model data), .BLEND (Blender), .UDIM (Polycount), .USD (Universal Scene Description), .VRML (Virtual Reality Modeling Language), .WebM (Audiovisual Media File Format), .X3D (ISO//IEC standard for declaratively representing 3D computer graphics), .3DS (Autodesk 3DS Max 3D Modeling Format), and .X_T (Parasolid). The facial scans may be imported into CAD in the same orientation and alignment performed in another program, as shown in FIGS. 17A-17C.

In some embodiments, various CAD modeling platforms may be used to receive the file type created, such as Autodesk AutoCAD, Autodesk Inventor, Autodesk Fusion 360, Autodesk TinkerCAD, Dassault Systèmes CATIA, Dassault Systèmes SolidWorks, Siemens PLM, Rhino 3D, Parametric Technology Corporation Creo, and the like. Some platforms may allow direct import of the file type into the software, while others may require an indirect approach using a predetermined location on a file directory.

Once the aligned facial scans are imported into a CAD program, as shown in FIGS. 17A-17C, measurements may be taken to calculate a space between the posterior aspect of the eyeglasses and the anterior aspect of the solid body. This process allows for the creation of a pair of eyeglasses comprising seals between some parts of the frame and the individual's face and some voids between parts of the frame and the individual's face.

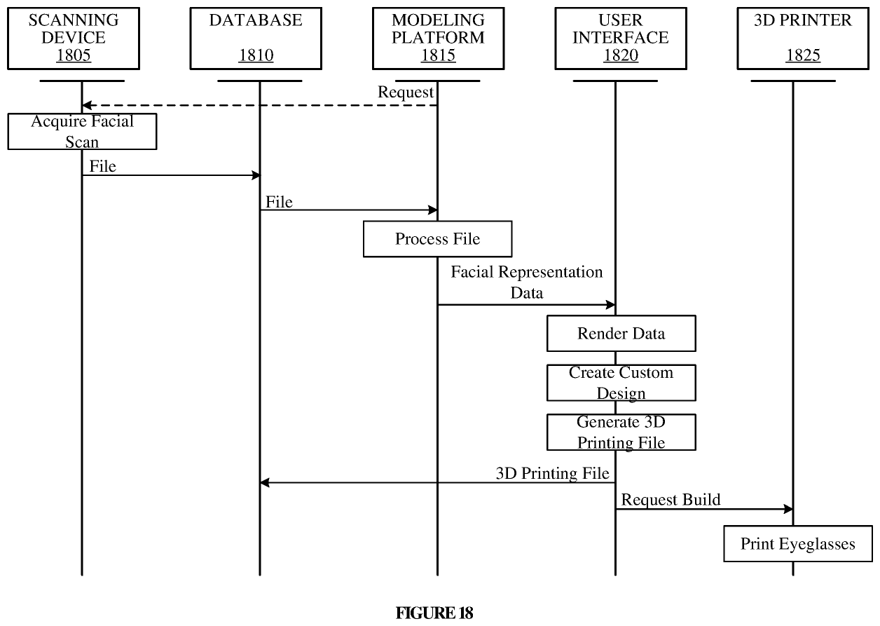

FIG. 18 is a sequence diagram illustrating an example of a set of communications between various components of a system that may be used in accordance with various embodiments of the present technology. As illustrated in FIG. 18, scanning device 1805 can acquire a facial scan of a user. In some embodiments, scanning device may operate independently or may be under the control of modeling platform 1815 which can send a request to initiate the scan. The 3D file of the model of the face of the user can be stored in database 1810 and retrieved by modeling platform 1815. Modeling platform 1815 can process the file and facial representation data can be transmitted to a user interface 1820 which renders a model of the user's face and allow an operator to create a custom eyewear design and generate a 3D printing-capable file. The design and 3D printing file can be stored in database 1810 and a request may be made from user interface 1820 to build and print the eyewear (or portion thereof such as the ridge-like reliefs that can be attached to a pair of frames) from 3D printer 1825.

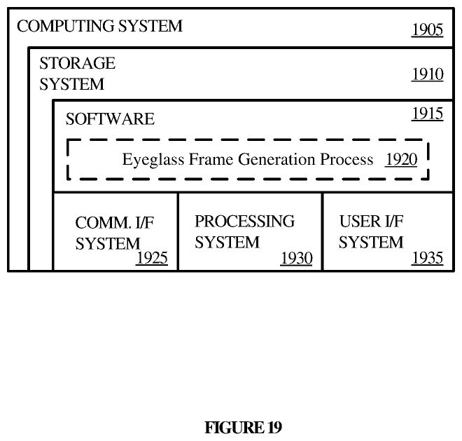

FIG. 19 illustrates computing system 1905 that is representative of any system or collection of systems in which the various processes, programs, services, and scenarios disclosed herein may be implemented. Examples of computing system 1905 include, but are not limited to, desktop computers, laptop computers, server computers, routers, web servers, cloud computing platforms, and data center equipment, as well as any other type of physical or virtual server machine, physical or virtual router, container, and any variation or combination thereof.

Computing system 1905 may be implemented as a single apparatus, system, or device or may be implemented in a distributed manner as multiple apparatuses, systems, or devices. Computing system 1905 includes, but is not limited to, processing system 1930, storage system 1910, software 1915, communication interface system 1925, and user interface system 1935 (optional). Processing system 1930 is operatively coupled with storage system 1910, communication interface system 1925, and user interface system 1635.

Processing system 1930 loads and executes software 1915 from storage system 1910. Software 1915 includes and implements process 1920, which is representative of the eyeglass frame generation process for dry eyes discussed with respect to the preceding Figures. When executed by processing system 1930 to provide eyeglass generation, software 1915 directs processing system 1930 to operate as described herein for at least the various processes, operational scenarios, and sequences discussed in the foregoing implementations. Computing system 1905 may optionally include additional devices, features, or functionality not discussed for purposes of brevity.

Referring still to FIG. 19, processing system 1930 may comprise a micro-processor and other circuitry that retrieves and executes software 1915 from storage system 1910. Processing system 1930 may be implemented within a single processing device but may also be distributed across multiple processing devices or sub-systems that cooperate in executing program instructions. Examples of processing system 1930 include general purpose central processing units, graphical processing units, application specific processors, and logic devices, as well as any other type of processing device, combinations, or variations thereof.

Storage system 1910 may comprise any computer readable storage media readable by processing system 1930 and capable of storing software 1915. Storage system 1910 may include volatile and nonvolatile, removable and non-removable media implemented in any method or technology for storage of information, such as computer readable instructions, data structures, program modules, or other data. Examples of storage media include random access memory, read only memory, magnetic disks, optical disks, optical media, flash memory, virtual memory and non-virtual memory, magnetic cassettes, magnetic tape, magnetic disk storage or other magnetic storage devices, or any other suitable storage media. In no case is the computer readable storage media a propagated signal.

In addition to computer readable storage media, in some implementations storage system 1910 may also include computer readable communication media over which at least some of software 1915 may be communicated internally or externally. Storage system 1910 may be implemented as a single storage device but may also be implemented across multiple storage devices or sub-systems co-located or distributed relative to each other. Storage system 1910 may comprise additional elements, such as a controller, capable of communicating with processing system 1930 or possibly other systems.

Software 1915 (including process 1920) may be implemented in program instructions and among other functions may, when executed by processing system 1930, direct processing system 1930 to operate as described with respect to the various operational scenarios, sequences, and processes illustrated herein. For example, software 1915 may include program instructions for implementing an eyeglass production process as described herein.

In particular, the program instructions may include various components or modules that cooperate or otherwise interact to carry out the various processes and operational scenarios described herein. The various components or modules may be embodied in compiled or interpreted instructions, or in some other variation or combination of instructions. The various components or modules may be executed in a synchronous or asynchronous manner, serially or in parallel, in a single threaded environment or multi-threaded, or in accordance with any other suitable execution paradigm, variation, or combination thereof. Software 1915 may include additional processes, programs, or components, such as operating system software, virtualization software, or other application software. Software 1915 may also comprise firmware or some other form of machine-readable processing instructions executable by processing system 1930.

In general, software 1915 may, when loaded into processing system 1930 and executed, transform a suitable apparatus, system, or device (of which computing system 1905 is representative) overall from a general-purpose computing system into a special-purpose computing system customized to provide eyeglass modeling processes as described herein. Indeed, encoding software 1915 on storage system 1910 may transform the physical structure of storage system 1910. The specific transformation of the physical structure may depend on various factors in different implementations of this description. Examples of such factors may include, but are not limited to, the technology used to implement the storage media of storage system 1910 and whether the computer-storage media are characterized as primary or secondary storage, as well as other factors.

For example, if the computer readable storage media are implemented as semiconductor-based memory, software 1915 may transform the physical state of the semiconductor memory when the program instructions are encoded therein, such as by transforming the state of transistors, capacitors, or other discrete circuit elements constituting the semiconductor memory. A similar transformation may occur with respect to magnetic or optical media. Other transformations of physical media are possible without departing from the scope of the present description, with the foregoing examples provided only to facilitate the present discussion.

Communication interface system 1925 may include communication connections and devices that allow for communication with other computing systems (not shown) over communication networks (not shown). Examples of connections and devices that together allow for inter-system communication may include network interface cards, antennas, power amplifiers, RF circuitry, transceivers, and other communication circuitry. The connections and devices may communicate over communication media to exchange communications with other computing systems or networks of systems, such as metal, glass, air, or any other suitable communication media. The aforementioned media, connections, and devices are well known and need not be discussed at length here.

Communication between computing system 1905 and other computing systems (not shown), may occur over a communication network or networks and in accordance with various communication protocols, combinations of protocols, or variations thereof. Examples include intranets, internets, the Internet, local area networks, wide area networks, wireless networks, wired networks, virtual networks, software defined networks, data center buses and backplanes, or any other type of network, combination of network, or variation thereof. The aforementioned communication networks and protocols are well known and need not be discussed at length here.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the technology is not intended to be exhaustive or to limit the technology to the precise form disclosed above. While specific examples for the technology are described above for illustrative purposes, various equivalent modifications are possible within the scope of the technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel or may be performed at different times. Further any specific numbers noted herein are only examples: alternative implementations may employ differing values or ranges.

The teachings of the technology provided herein can be applied to other systems, not necessarily the system described above. The elements and acts of the various examples described above can be combined to provide further implementations of the technology. Some alternative implementations of the technology may include not only additional elements to those implementations noted above, but also may include fewer elements.

These and other changes can be made to the technology in light of the above Detailed Description. While the above description describes certain examples of the technology, and describes the best mode contemplated, no matter how detailed the above appears in text, the technology can be practiced in many ways. Details of the system may vary considerably in its specific implementation, while still being encompassed by the technology disclosed herein. As noted above, particular terminology used when describing certain features or aspects of the technology should not be taken to imply that the terminology is being redefined herein to be restricted to any specific characteristics, features, or aspects of the technology with which that terminology is associated. In general, the terms used in the following claims should not be construed to limit the technology to the specific examples disclosed in the specification, unless the above Detailed Description section explicitly defines such terms. Accordingly, the actual scope of the technology encompasses not only the disclosed examples, but also all equivalent ways of practicing or implementing the technology under the claims.

To reduce the number of claims, certain aspects of the technology are presented below in certain claim forms, but the applicant contemplates the various aspects of the technology in any number of claim forms. For example, while only one aspect of the technology is recited as a computer-readable medium claim, other aspects may likewise be embodied as a computer-readable medium claim, or in other forms, such as being embodied in a means-plus-function claim. Any claims intended to be treated under 35 U.S.C. § 112(f) will begin with the words “means for,” but use of the term “for” in any other context is not intended to invoke treatment under 35 U.S.C. § 112(f). Accordingly, the applicant reserves the right to pursue additional claims after filing this application to pursue such additional claim forms, in either this application or in a continuing application.

What is claimed is:

1. An eyewear apparatus for management of dry eyes, the eyewear apparatus comprising:
a frame having a front that includes a first eyepiece to securely affix a right lens and a second eyepiece to securely affix a left lens; and
a ridge-like relief custom shaped to a contour of facial features of an individual to create a seal between a portion of the frame and a face of a user, wherein the seal is configured to fill a void between a superior aspect of the frame and the face, fill a void between an inferior aspect of the frame and the face, and fill a void between a nasal region of the frame and the face.

2. The eyewear apparatus of claim 1, wherein the seal, at least in part, maintains a minimum humidity level of forty percent and a maximum humidity level of ninety percent between the eyewear apparatus and the face.

3. The eyewear apparatus of claim 1, wherein a right temporal aspect of the frame and a left temporal aspect of the frame are not sealed such that the right temporal aspect and the left temporal aspect allow an airflow between the eyewear apparatus and the face.

4. The eyewear apparatus of claim 1, wherein the ridge-like relief is built separately from the frame and wherein the frame includes one or more attachment mechanisms allowing the ridge-like relief to be securely affixed to the frame.

5. The eyewear apparatus of claim 1 wherein the frame and the seal are custom designed for the face of the user.

6. A method of designing and creating a customized eyeglass frame to relieve symptoms of dry eyes, the method comprising:
acquiring a scan of at least a portion of a face of an individual;
converting the scan of the at least a portion of the face into a three-dimensional computer model of the face;
generating a computer model of an eyewear apparatus, wherein the eyewear apparatus comprises:
a frame;
a seal between a superior portion of the frame and the three-dimensional computer model of the face;
a seal between an inferior portion of the frame and the three-dimensional computer model of the face; and
a seal between a nasal portion of the frame and the three-dimensional computer model of the face; and
converting the computer model of the eyewear apparatus into a pair of eyeglasses via a three-dimensional printer.

7. The method of claim 6, wherein converting the scan of the at least a portion of the face into a three-dimensional model of the face comprises:
generating a surface mesh of at least a portion of the face based on the scan;
converting the surface mesh into a solid body model; and
importing the solid body model into the three-dimensional computer model of the face.

8. The method of claim 6, wherein:
a right temporal void between a right temporal aspect of the frame and the face is not sealed;
a left temporal void between a left temporal aspect of the frame and the face is not sealed; and
the right temporal void and the left temporal void allow an airflow between the eyewear apparatus and the face.

9. The method of claim 6, wherein the seal, at least in part, maintains a minimum humidity level of 40 percent and a maximum humidity level of 90 percent between the eyewear apparatus and the face.

10. The method of claim 6, wherein the frame and each of the seals are custom designed for the face of a user.

11. One or more non-transitory computer-readable storage media having program instructions stored thereon to facilitate creation of eyewear for dry eyes, wherein the program instructions, when executed by a computing system, direct the computing system to at least:
receive a three-dimensional scan of at least a portion of a face;
generate a face model of at least a portion of the face based on the three-dimensional scan;
generate a model of a seal, wherein the seal fills a void between:
a superior portion of an eyeglass frame and the face;
an inferior portion of the eyeglass frame and the face; and
a nasal portion of the eyeglass frame and the face; and
send the model of the seal to a three-dimensional printer to be printed.

12. The one or more non-transitory computer-readable storage media of claim 11, wherein a right temporal portion of the eyeglass frame and a left temporal portion of the eyeglass frame are not sealed such that the right temporal portion and the left temporal portion allow an airflow between the eyeglass frame and the face.

13. The one or more non-transitory computer-readable storage media of claim 11, wherein the seal, at least in part, maintains a minimum humidity level of 40 percent and a maximum humidity level of 90 percent between the eyeglass frame and the face.

14. The one or more non-transitory computer-readable storage media of claim 11, wherein the eyeglass frame comprises one or more receiving components, the seal comprises one or more attachment components, and the seal can be attached and detached from the eyeglass frame.

15. The one or more non-transitory computer-readable storage media of claim 11, wherein the face model of at least a portion of the face is a three-dimensional rendering of the face.

16. The one or more non-transitory computer-readable storage media of claim 11, wherein the frame and each of the seals are custom-designed for the face of a user.

17. A system for creating custom eyewear, the system comprising:
a three-dimensional facial scanner that generates a facial scan of at least a portion of a face;
one or more computer-readable storage media;
at least one processing system operatively coupled with the one or more computer-readable storage media having program instructions stored thereon that, when executed by the at least one processing system, direct the processing system to:
receive the facial scan of at least a portion of the face;
generate a model of at least a portion of the face based on the facial scan;
generate a model of a seal, wherein the seal fills a void between each of:
a superior aspect of an eyeglass frame and the face;
an inferior aspect of the eyeglass frame and the face; and
a nasal aspect of the eyeglass frame and the model the face; and
send the model of the seal to a three-dimensional printer to be printed; and
the three-dimensional printer, wherein the three-dimensional printer prints at least the seal.

18. The system of claim 17, wherein the three-dimensional printer further prints the eyeglass frame.

19. The system of claim 17, wherein the seal does not fill a void between each of:
a right temporal void, wherein the right temporal void is between a right temporal aspect of the eyeglass frame and the face; and
a left temporal void, wherein the left temporal void is between a left temporal aspect of the eyeglass frame and the face.

20. The system of claim 19, wherein the right temporal void and the left temporal void allow an airflow between the eyeglass frame and the face, and the airflow, at least in part, prevents a humidity level between the eyeglass frame and the face from exceeding 90 percent.
